(19) United States
(12) Patent Application Publication
(10) Pub. No.: US 2021/0383534 A1
(43) Pub. Date: Dec. 9, 2021

(54) SYSTEM AND METHODS FOR IMAGE SEGMENTATION AND CLASSIFICATION USING REDUCED DEPTH CONVOLUTIONAL NEURAL NETWORKS

(71) Applicant: GE Precision Healthcare LLC, Milwaukee, WI (US)

(72) Inventor: Rimon Tadross, Milwaukee, WI (US)

(21) Appl. No.: 16/891,628

(22) Filed: Jun. 3, 2020

(51) Int. Cl. G06T 7/00; G06T 7/11; G06N 3/04; G06K 9/62; G06K 9/32

(52) U.S. Cl. CPC G06T 7/0012; G06T 7/11; G06N 3/0481; G06T 2207/20081; G06K 9/3233; G06T 2207/20084; G06K 9/6267

(57) ABSTRACT

Methods and systems are provided for segmenting and/or classifying images using convolutional neural networks (CNNs). In one embodiment, a method comprises, receiving an image having a first size, downsampling the image to produce a downsampled image of a pre-determined size, wherein the pre-determined size is less than the first size, feeding the downsampled image to a CNN, wherein a first convolutional layer of the CNN comprises a first plurality of convolutional filters, each of the first plurality of convolutional filters having a receptive field size larger than a threshold receptive field size, identifying one or more anatomical structures of the downsampled image using the first plurality of convolutional filters; and mapping the one or more anatomical structures to a segmentation map or image classification using one or more subsequent layers of the CNN. In this way, a number of encoding layers of the trained CNN may be substantially reduced.

### TECHNICAL FIELD

Embodiments of the subject matter disclosed herein relate to image processing using convolutional neural networks, and more particularly, to systems and methods of segmenting and/or classifying medical images using convolutional neural networks of reduced depth.

### BACKGROUND

Medical imaging systems are often used to obtain internal physiological information of a subject, such as a patient. For example, a medical imaging system may be used to obtain images of the bone structure, the brain, the heart, the lungs, and various other features of a patient. Medical imaging systems may include magnetic resonance imaging (MRI) systems, computed tomography (CT) systems, x-ray systems, ultrasound systems, and various other imaging modalities.

Analysis and processing of medical images increasingly includes segmentation of anatomical regions of interest and/or image classification using machine learning models. One such approach for segmenting and/or classifying medical images includes identifying features present within a medical image using a plurality of convolutional layers of a convolutional neural network (CNN), and mapping the identified features to a segmentation map or image classification. As an example, an MRI image of an organ of interest may be acquired, and the regions of the image including the organ of interest may be automatically labeled/segmented in a segmentation map produced by a trained CNN. In another example, an image of an abdomen of a patient may be classified as an abdominal image by identifying one or more features of the image using one or more convolutional layers, and passing the identified features to a classification network configured to output a most probable image classification for the medical image from a finite list of pre-determined image classification labels.

One drawback associated with conventional CNNs is the large number of convolutional layers needed to identify anatomical regions of interest and/or to classify a medical image. As an example, one limitation of deep CNNs is the vanishing gradient phenomena, encountered when attempting to train conventional CNNs, wherein the gradient of the cost/loss function, used to learn convolutional filter weights diminishes with each layer of the CNN, which may result in slow and computationally intensive training for “deep” networks. A related limitation of conventional CNNs is the large parameter space which is to be optimized during training, as the number of convolutional filter weights to be optimized increases with each additional convolutional layer, and the probability of converging to a local optimum, increases with the number of parameters to be optimized. Conventional CNNs, which may comprise hundreds of thousands to millions of parameters, may consume substantial computational resources, both during training and during implementation. This may result in long training times, and slow medical image analysis. Further, conventional CNNs may perform particularly poorly when attempting to segment regions of interest which occupy a relatively large fraction of a medical image (e.g., greater than 20% of the area of a medical image), or when attempting to determine an image classification (which may rely on information from spatially distant portions of the medical image), as conventional convolutional filters comprise receptive fields occupying a small fraction of the image, and therefore such segmentation/classification relies on the CNN to “learn” the correct assemblage of relatively small features into the desired larger composite features.

### SUMMARY

The inventors herein have identified systems and methods for image segmentation and classification using CNNs of reduced encoder depth, which may produce accurate segmentation maps of high resolution and image classifications of high accuracy, without consuming the computational resources or time of conventional CNNs. In one embodiment, a segmentation map or image classification may be produced by a method comprising, receiving an image having a first size, downsampling the image to produce a downsampled image of a pre-determined size, wherein the pre-determined size is less than the first size, feeding the downsampled image to a convolutional neural network (CNN), wherein a first convolutional layer of the CNN comprises a first plurality of convolutional filters, each of the first plurality of convolutional filters having a receptive field size larger than a threshold receptive field size, identifying one or more anatomical structures of the downsampled image using the first plurality of convolutional filters, and mapping the one or more anatomical structures to a segmentation map or image classification using one or more subsequent layers of the CNN. By providing a first convolutional layer of a CNN with a plurality of filters having receptive fields larger than a threshold size, larger/more complex features may be identified by the first convolutional layer, without relying on a deep encoder. Further, by downsampling the image prior to segmentation/classification, larger convolutional filters and more convolutional filters, may be used in the first convolutional layer, without substantially increasing the number of parameters of the first convolutional layer compared to conventional CNNs.

In this way, it is possible to reduce the number of convolutional layers/parameters in CNNs, while maintaining accuracy of segmentation/classification, as CNNs comprising a reduced number of convolutional layers/parameters may be trained and implemented more rapidly than conventional CNNs, and further, a probability of said CNNs learning a set of locally optimal (and not globally optimal) parameters may be decreased.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an exemplary embodiment of an image processing system;

FIG. 2 shows one embodiment of an image segmentation system comprising a reduced depth CNN;

FIG. 3 shows a flowchart of an exemplary method for segmenting medical images using a reduced depth CNN;

FIG. 4 shows a flowchart of an exemplary method for determining an image classification using a reduced depth CNN;

FIG. 5 shows a flowchart of a first exemplary method for refining region of interest boundaries in a segmentation map produced by a reduced depth CNN;

FIG. 6 shows a flowchart of a second exemplary method for refining region of interest boundaries in a segmentation map produced by a reduced depth CNN;

FIG. 7 shows a flowchart of an exemplary method for training a reduced depth CNN;

FIG. 8 illustrates an exemplary embodiment of the first method for refining region of interest boundaries in a segmentation map; and

FIG. 9 illustrates an exemplary embodiment of the second method for refining region of interest boundaries of a segmentation map.

The drawings illustrate specific aspects of the described systems and methods for determining segmentation maps and/or image classifications for medical images using reduced depth CNNs. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems and methods.

### DETAILED DESCRIPTION

The following description relates to systems and methods for segmenting and/or classifying medical images using CNNs of reduced depth. Conventional CNN architectures include a plurality of convolutional layers configured to detect features present in an input image (the plurality of convolutional layers also referred to as an encoder or an encoding portion of the CNN), and a subsequent plurality of layers configured to map said identified features to one or more outputs, such as a segmentation map or image classification. Each convolutional layer comprises one or more convolutional filters, and each convolutional filter is “passed over”/receives input from, each sub-region of an input image, or preceding feature map, to identify pixel intensity patterns and/or feature patterns, which match the learned weights of the convolutional filter. The size of the sub-region of the input image, or preceding feature map, from which a convolutional filter receives input is referred to as the kernel size or the receptive field size of the convolutional filter. Convolutional filters with smaller receptive field sizes are limited to identifying relatively small features (e.g., lines, edges, corners), whereas convolutional filters with larger receptive fields (or convolutional filters located at deeper layers of the encoding portion of the CNN) are able to identify larger features/composite features (e.g., eyes, noses, faces, etc.).

Conventional CNNs used in medical image segmentation and/or classification comprise relatively deep encoding portions, generally including five or more convolutional layers, wherein each of the convolutional layers include convolutional filters of relatively small receptive field size (e.g., 3×3 pixels/feature channels, which corresponds to approximately 0.0137% of the area of a conventional 256×256 input image). In conventional approaches, input images are 256×256, a standard size in the art of image processing, although in some applications images of larger sizes may be used. Images smaller than 256×256 are conventionally not used in neural network based image processing, as information content of an image may decrease with decreasing resolution. In conventional CNNs, relatively shallow convolutional layers (e.g., a first convolutional layer) extract atomic/elemental features such as lines and edges, whereas deeper convolutional layers extract composite features representing combinations of features identified/extracted by previous layers, e.g., a first convolutional layer identifying corners and lines in an image, and a second convolutional layer identifying squares and triangles in the image based on combinations/patterns of the previously identified corners and lines. Conventional CNNs use “deep” networks (e.g., networks comprising 5 or more convolutional layers) wherein receptive field sizes of the convolutional filters in the first convolutional layer are relatively small, e.g., 3×3. Conventional CNNs have shown poor performance on segmentation of regions of interest (ROIs) occupying a relatively large portion of an image (e.g., greater than 25%) and image classification tasks involving classifying an entire image based on the overall contents of the image. Said another way, conventional CNNs utilize convolutional filters with receptive fields substantially smaller than the images to be classified or the ROIs to be segmented, and thus rely on the CNN to learn how to synthesize the relatively small spatial features extracted by the first convolutional layer into the larger features to be labeled/segmented, such as an ROI, or an image classification based on contents of an entire image. Depending on the later convolutional layers of a network to combine the small spatial features extracted by the earlier convolutional layers of the network is very sensitive to the choice of the network architecture and to the training procedure, and is further prone to having the network parameters converge to a local minimum during training, because using larger and deeper networks means much larger number of network parameters and a subsequently higher dimensional loss landscape to search during training. A further drawback to using deep CNNs with large numbers of parameters is the time and computational resources used during both training and implementation.

The inventors herein have identified systems and methods which may at least partially address the above identified issues. In one embodiment, a method for segmenting and/or classifying an image comprises, receiving an image having a first size, downsampling the image to produce a downsampled image of a pre-determined size, wherein the pre-determined size is less than the first size, feeding the downsampled image to a trained CNN, wherein a first convolutional layer of the trained CNN comprises a first plurality of convolutional filters, each of the plurality of convolutional filters having a receptive field size larger than a threshold size, identifying one or more features of the downsampled image using the first plurality of convolutional filters, and mapping the one or more features to a segmentation map or image classification using one or more subsequent layers of the trained CNN. By setting the receptive field size threshold based on the ROIs to be segmented and/or the image size, richer spatial relationships of features may be identified earlier on in the CNN, enabling more efficient identification of ROIs and/or large image features correlated with holistic image classification. In one embodiment, a first convolutional layer of a CNN may comprise a plurality of convolutional filters having receptive field sizes from 6% to 100% (and any amount therebetween) of the size of a downsampled input image. Further, downsampling the image prior to feeding the image to the trained CNN enables use of convolutional filters of larger receptive field size relative to the input image size, and/or use of a larger number of convolutional filters, without a concomitant increase in computational complexity, training time, implementation time, etc.

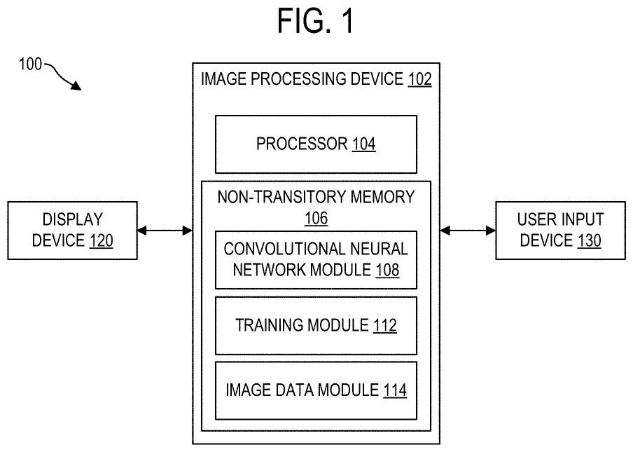

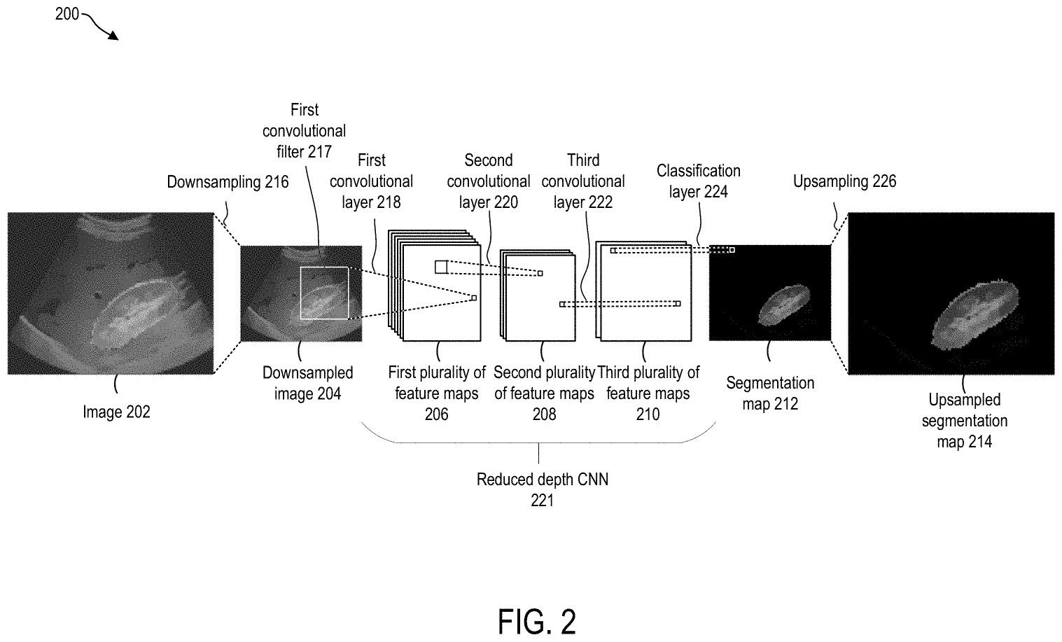

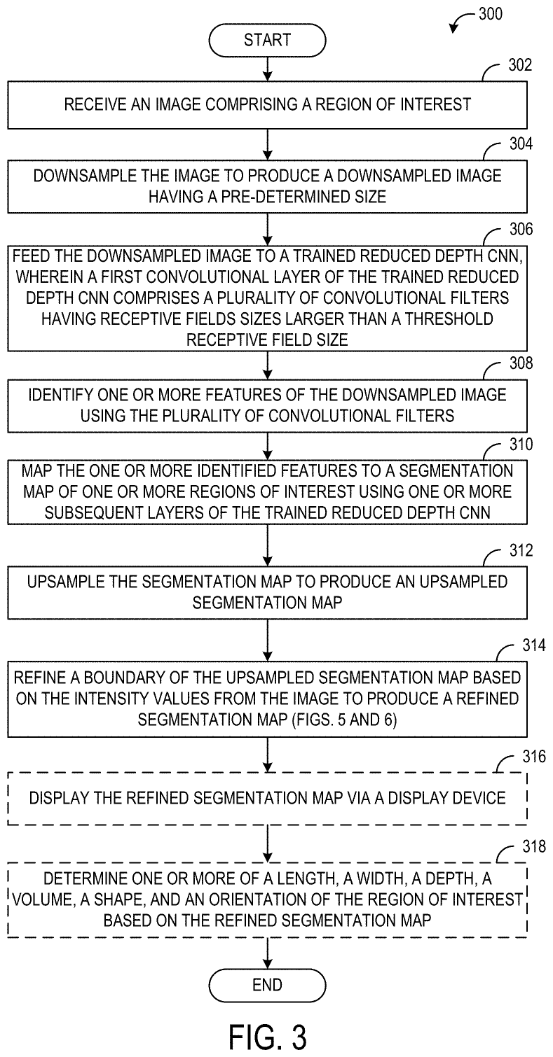

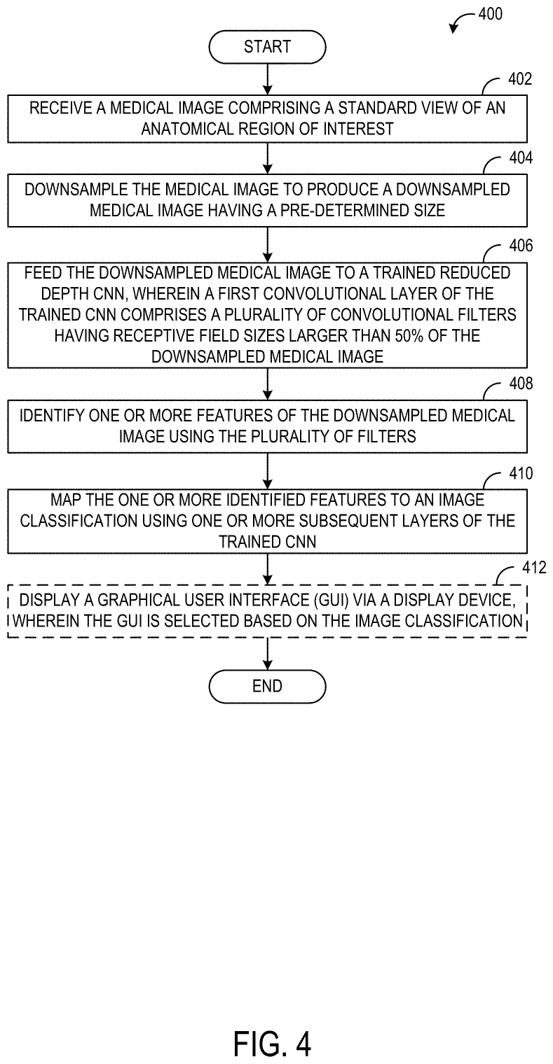

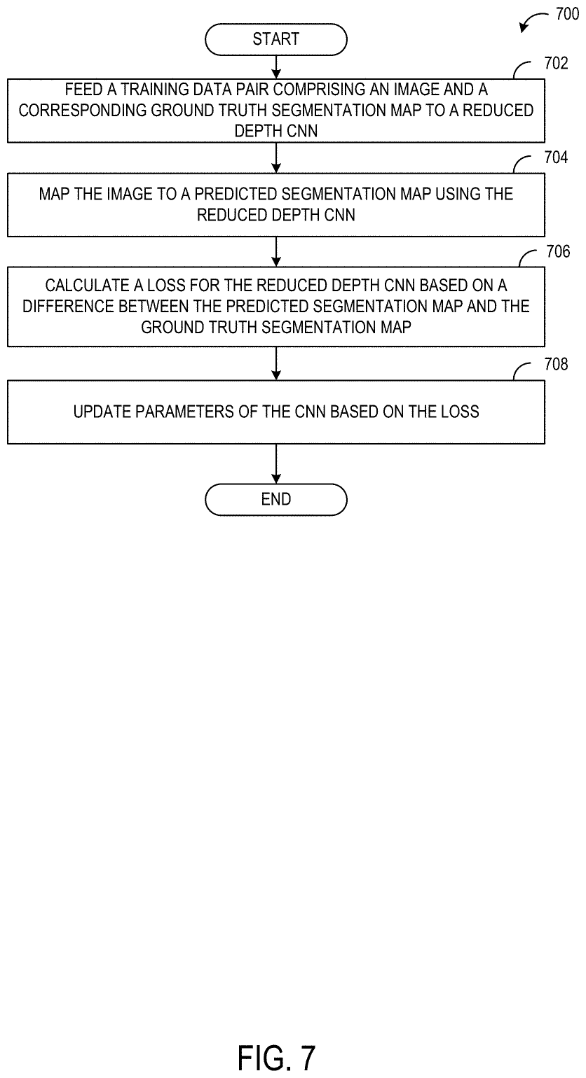

In one embodiment, image processing system 100, shown in FIG. 1, may store one or more trained reduced depth CNNs in convolutional neural network module 108. The trained CNNs stored in the convolutional neural network module 108 may be trained according to one or more steps of method 700, shown in FIG. 7. Image processing system 100 may receive and process images acquired via various imaging modalities, such as MRI, X-ray, ultrasound, CT, etc., and may determine a segmentation map for one or more ROIs present within said images, and/or determine a standard view classification of the one or more images. In particular, image processing system 100 may implement method 300, shown in FIG. 3, to produce a segmentation map of one or more ROIs present within an image, using an image segmentation system 200, illustrated in FIG. 2. Image processing system 100 may likewise determine an image classification for the image using one or more operations of method 400, shown in FIG. 4. Segmentation maps produced according to one or more operations of method 300 may be further be processed according to one or more operations of methods 500 and/or 600, to refine ROI boundaries of the one or more ROIs identified therein. The ROI boundary refining approaches of methods 500 and 600 are illustrated in FIGS. 800 and 900, respectively.

Turning now to FIG. 1, image processing system 100 is shown, in accordance with an exemplary embodiment. In some embodiments, image processing system 100 is incorporated into an imaging system, such as a medical imaging system. In some embodiments, at least a portion of image processing system 100 is disposed at a device (e.g., edge device, server, etc.) communicably coupled to a medical imaging system via wired and/or wireless connections. In some embodiments, at least a portion of the image processing system 100 is disposed at a device (e.g., a workstation), located remote from a medical imaging system, which is configured to receive images from the medical imaging system or from a storage device configured to store images acquired by the medical imaging system. Image processing system 100 may comprise image processing device 102, user input device 130, and display device 120. In some embodiments, image processing device 102 may be communicably coupled to a picture archiving and communication system (PACS), and may receive images from, and/or send images to, the PACS.

Image processing device 102 includes a processor 104 configured to execute machine readable instructions stored in non-transitory memory 106. Processor 104 may be single core or multi-core, and the programs executed thereon may be configured for parallel or distributed processing. In some embodiments, the processor 104 may optionally include individual components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration.

Non-transitory memory 106 may store convolutional neural network module 108, training module 112, and image data 114. Convolutional neural network module 108 may include one or more trained or untrained convolutional neural networks, comprising a plurality of weights and biases, activation functions, pooling functions, and instructions for implementing the one or more convolutional neural networks to segment ROIs and/or determine image classifications for various images, including 2D and 3D medical images. In some embodiments, convolutional neural network module 108 may comprise reduced depth CNNs comprising less than 5 convolutional layers, and may determine a segmentation map and/or image classification for an input medical image using said one or more reduced depth CNNs by executing one more operations of methods 300 and/or 400.

Convolutional neural network module 108 may include various metadata pertaining to the trained and/or un-trained CNNs. In some embodiments, the CNN metadata may include an indication of the training data used to train a CNN, a training method employed to train a CNN, and an accuracy/validation score of a trained CNN. In some embodiments, convolutional neural network module 108 may include metadata indicating the type(s) of ROI for which the CNN is trained to produce segmentation maps, a size of input image which the trained CNN is configured to process, and a type of anatomy, and/or a type of imaging modality, to which the trained CNN may be applied. In some embodiments, the convolutional neural network module 108 is not disposed at the image processing device 102, but is disposed at a remote device communicably coupled with image processing device 102 via wired or wireless connection.

Non-transitory memory 106 further includes training module 112, which comprises machine executable instructions for training one or more of the CNNs stored in convolutional neural network module 108. In some embodiments, training module 112 may include instructions for training a reduced depth CNN according to one or more of the operations of method 700, shown in FIG. 7, and discussed in more detail below. In one embodiment, the training module 112 may include gradient descent algorithms, loss/cost functions, and machine executable rules for generating and/or selecting training data for use in training reduced depth CNNs. In some embodiments, the training module 112 is not disposed at the image processing device 102, but is disposed remotely, and is communicably coupled with image processing device 102.

Non-transitory memory 106 may further include image data module 114, comprising images/imaging data acquired by one or more imaging devices, including but not limited to, ultrasound images, MRI images, PET images, X-ray images, CT images. The images stored in image data module 114 may comprise medical images from various imaging modalities or from various makes/models of medical imaging devices, and may comprise images of various views of anatomical regions of one or more patients. In some embodiments, medical images stored in image data module 114 may include information identifying an imaging modality and/or an imaging device (e.g., model and manufacturer of an imaging device) by which the medical image was acquired. In some embodiments, images stored in image data module 114 may include metadata indicating one or more acquisition parameters used to acquire said images. In one example, metadata for the images may be stored in DICOM headers of the images. In some embodiments, image data module 114 may comprise x-ray images acquired by an x-ray device, MR images captured by an MRI system, CT images captured by a CT imaging system, PET images captured by a PET system, and/or one or more additional types of medical images.

In some embodiments, the non-transitory memory 106 may include components disposed at two or more devices, which may be remotely located and/or configured for coordinated processing. In some embodiments, one or more aspects of the non-transitory memory 106 may include remotely-accessible networked storage devices configured in a cloud computing configuration.

Image processing system 100 may further include user input device 130. User input device 130 may comprise one or more of a touchscreen, a keyboard, a mouse, a trackpad, a motion sensing camera, or other device configured to enable a user to interact with and manipulate data within image processing system 100. In some embodiments, user input device 130 enables a user to select one or more types of ROI to be segmented in a medical image.

Display device 120 may include one or more display devices utilizing virtually any type of technology. In some embodiments, display device 120 may comprise a computer monitor, a touchscreen, a projector, or other display device known in the art. Display device 120 may be configured to receive data from image processing device 102, and to display a segmentation map of a medical image showing a location of one or more regions of interest. In some embodiments, image processing device 102 may determine a standard view classification of a medical image, may select a graphical user interface (GUI) based on the standard view classification of the image, and may display via display device 120 the medical image and the GUI. Display device 120 may be combined with processor 104, non-transitory memory 106, and/or user input device 130 in a shared enclosure, or may be a peripheral display device and may comprise a monitor, touchscreen, projector, or other display device known in the art, which may enable a user to view images, and/or interact with various data stored in non-transitory memory 106.

It should be understood that image processing system 100 shown in FIG. 1 is for illustration, not for limitation. Another appropriate image processing system may include more, fewer, or different components.

Turning to FIG. 2, a box diagram of an image segmentation system 200, for producing segmentation maps from medical images using a reduced depth CNN, is shown. Image segmentation system 200 may be implemented by an image processing system, such as image processing system 100, or other appropriately configured computing systems. Image segmentation system 200 is configured to receive one or more images, such as image 202, comprising a region of interest, and map the one or more received images to one or more corresponding segmentation maps, such as upsampled segmentation map 214, using a reduced depth CNN 221. In some embodiments, segmentation system 200 may be configured to segment one or more anatomical regions of interest, such as a kidney, blood vessels, tumors, etc., or non-anatomical regions of interest such as traffic signs, text, vehicles, etc.

Image 202 may comprise a two-dimensional (2D) or three-dimensional (3D) array of pixel intensity values in one or more color channels, or a time series of 2D or 3D arrays of pixel intensity values. In some embodiments, image 202 comprises a greyscale/grey-level image. In some embodiments, image 202 comprises a colored image comprising two or more color channels. Image 202 may comprise a medical image or non-medical image and may comprise an anatomical or non-anatomical region of interest. In some embodiments, image 202 may not include a region of interest. Segmentation system 200 may receive image 202 from a medical imaging device or other imaging device via wired or wireless connection, and may further receive metadata associated with image 202 indicating one or more acquisition parameters of image 202, including an indication of an imaging modality used to acquire image 202, an indication of imaging device settings used to acquire image 202, an indication of field-of-view (FOV) data, etc.

Image 202 may be of a first size/resolution, that is, may comprise a matrix/array of pixel intensity values having a first number of data points. The first size/resolution may be a function of the imaging modality and imaging settings used to acquire image 202, and/or a file format used to store image 202. As indicated by downsampling 216, image 202 is downsampled from the first size to a downsampled image 204 having a second, smaller size/resolution. The second size/resolution may be pre-determined based on a desired number of input nodes/neurons to be used in reduced depth CNN 221. In some embodiments, the number of pixel intensity values of image 202 may be reduced by downsampling 216 to a pre-determined number of pixel intensity values, wherein the pre-determined number of pixel intensity values correspond to a number of input nodes/neurons in reduced depth CNN. The desired number of input nodes/neurons may in turn be selected based on a desired receptive field size of convolutional filters in first convolutional layer 218, and/or a desired total number of convolutional filters in first convolutional layer 218, such that as the receptive field size of the convolutional filters in the first convolutional layer 218 increase or as the total number of convolutional filters in first convolutional layer 218 increases, the number of input nodes/neurons (and therefore the second size) is reduced, thereby maintaining a total number of network parameters, below a threshold number of parameters. The number of network parameters is correlated with computational complexity and implementation time, ergo, by maintaining the total number of network parameters below the threshold number of network parameters, the computational complexity and/or implementation time is controlled to be within a pre-determined range.

Downsampling 216 may comprise one or more data pooling or downsampling operations, including one or more of max pooling, decimation, average pooling, and compression. In some embodiments, downsampling 216 may include a dynamic determination of a downsampling ratio, wherein the downsampling ratio is determined based on a ratio of the first size of image 202 to the pre-determined second size of downsampled image 204, wherein as the ratio of the first size to the second size increases, the downsampling ratio also increases. Downsampling 216 produces downsampled image 204 from image 202.

Downsampled image 204 comprises a downsampled/compressed image, wherein a size/resolution of downsampled image 204 equals the pre-determined size, also referred to herein as the second size. Selection of the pre-determined size may be based on a desired number of parameters/weights of reduced depth CNN 221, as discussed above. By downsizing image 202 to produce downsampled image 204, a receptive field of one or more convolutional filters, such as first convolutional filter 217, of first convolutional layer 218, may occupy greater than a threshold area of one or more regions of interest, without incurring a substantial reduction in computational efficiency or a substantial increase in implementation time. ROIs present in image 202 may occupy a smaller number of pixels in downsampled image 204 than in image 202. As an example, an image of an organ within image 202 may occupy an area of 400 pixels, and upon downsampling image 202 using a downsampling ratio of 2, a downsampled image of the organ within downsampled image 204 may occupy 100 pixels, a reduction in convolutional filter weights of 75%. In this way, a convolutional filter having a receptive field size of 100 pixels may cover a majority of the organ (as imaged in downsampled image 204) without employing a convolutional filter with a receptive field size of 400 pixels. Thus, by downsampling image 202, convolutional filters occupying a larger portion of an ROI may be employed in a first convolutional layer 218 of the reduced depth CNN 221, enabling said convolutional filters to cover greater than a threshold area of one or more regions of interest, without a proportionate increase in the number of convolutional filter weights.

Downsampled image 204 may be fed to an input layer of reduced depth CNN 221, and propagated/mapped to a segmentation map of one or more ROIs within downsampled image 204, such as segmentation map 212. Reduced depth CNN 221 is shown comprising a first convolutional layer 218, comprising a first plurality of convolutional filters including first convolutional filter 217, a second convolutional layer 220 comprising a second plurality of convolutional filters, a third convolutional layer 222 comprising a third plurality of convolutional filters, and a classification layer 224 configured to receive features extracted by the convolutional layers and produce segmentation map 212 therefrom. Reduced depth CNN may further comprise additional non-convolutional layers, such as an input layer, output layer, fully-connected layer, etc., however reduced depth CNN 221 is illustrated in FIG. 2 to emphasize the number and arrangement of convolutional layers therein.

The first convolutional layer 218 maps image data (e.g., pixel intensity data) from downsampled image 204 to first plurality of feature maps 206, comprising one or more extracted/identified features produced by the first plurality of convolutional filters of the first convolutional layer 218. Each convolutional filter of the first plurality of convolutional filters comprises a receptive field size greater than a pre-determined receptive field size threshold. The receptive field size threshold may be pre-determined based on the pre-determined second size of downsampled image 204, and further based on an expected relative coverage/shape of one or more regions of interest to be segmented. In some embodiments, the receptive field size threshold may be set to 20% or more of an expected size of an anatomical structure (e.g., blood vessels, femur, organ, etc.). In one embodiment, if a region of interest is expected to occupy 25% of the area/volume of an image, the threshold receptive field size of convolutional filters within the first convolutional layer 218 may be set to 0.25×A×B, where A is the pre-determined second size of downsampled image 204, and B is a desired relative coverage of the ROI. In some embodiments, a desired relative coverage of an ROI may be 20% or more. In a particular example, if downsampled image 204 comprises a 100×100 matrix of pixel intensity values, a region of interest to be segmented is expected to occupy 30% of image 202, and the receptive field is desired to cover 80% of the region of interest then the receptive field size threshold may be set to (100×100)×0.30×0.8=2,400 pixels.

The shape of the first plurality of convolutional filters may further be set based on an expected shape of the ROI to be segmented. In one example, if the ROI to be segmented comprises a substantially oblong shape, the shape of the receptive fields of the first plurality of convolutional filters may be set to a rectangle (in the case of 2D images) or rectangular solid (in the case of 3D images). If the shape of the ROI to be segmented comprises one or more axes of symmetry, or comprises one or more repeating subunits, the shape and size of the receptive fields of the first plurality of convolutional filters may be set based thereon, to leverage the symmetry or modular nature of an ROI. In one embodiment, if the ROI to be segmented comprises blood vessels, which are substantially greater in length than in width/diameter, a convolutional filters in the first convolutional layer 218 of reduced depth CNN 221 may comprise receptive fields with a width of several pixels (e.g., 1 to 5 pixels), and a length set based on an estimate of the width of the blood vessels to be segmented, thereby enabling the receptive field to cover at least a majority of the width of the blood vessels, without needing to cover a majority of the length of the blood vessels. As can be seen in FIG. 2, first convolutional filter 217 occupies a majority of the region of interest to be segmented, and comprises a square shape, as the ROI to be segmented comprises a substantially oblong shape.

A number of the first plurality of convolutional filters may be set based on an expected variation in shape/size of the ROI to be segmented. In general, as the receptive field sizes of the first plurality of convolutional filters increases, the number of the first plurality of convolutional filters may also increase, to account for the increased range of possible shapes/sizes of features which may be identified/extracted thereby. An advantage of employing convolutional filters in a first convolutional layer 218 having relatively large receptive field sizes, is that a depth of reduced depth CNN 221 may be reduced, as small features are no longer aggregated through numerous convolutional layers to form larger features, which are then used to segment an ROI. Instead, features comprising substantial portions of an ROI (e.g., greater than 25%) are identified in a first convolutional layer 218, using convolutional filters having receptive field sizes covering a majority of an extent of an ROI in at least one dimension. As the receptive field size threshold increases the number of the first plurality of convolutional filters may also increase to account for the diversity in shape/size of feature which may be identified via the first plurality of convolutional filters. Said another way, as reduced depth CNN 221 is configured to identify larger features in a first convolutional layer than are conventionally detected in a first convolutional layer of a CNN, the range of variation of said features may also be larger than the range of variation in features detected in a first layer of a conventional CNN, and therefore a greater number of convolutional filters may be employed in the first convolutional layer 218 than in the subsequent layers 220, 222, and 224. Likewise, the receptive field sizes of the first plurality of convolutional filters in the first convolutional layer 218 may be larger than the receptive field sizes of convolutional filters in the subsequent convolutional filters 220 and 222.

First plurality of feature maps 206 comprise a plurality of output values from first convolutional layer 218, wherein each output value corresponds to a degree of match between one or more convolutional filters in the first convolutional layer 218 with the pixel intensity data of downsampled image 204. Each distinct filter in first convolutional layer 218 may produce a distinct feature map in first plurality of feature maps 206. The first convolutional layer 218 identifies/extracts features from downsampled image 204, and for each convolutional filter of the first plurality of convolutional filters, a corresponding feature map is produced in first plurality of feature maps 206. In other words, the number of feature maps in first plurality of feature maps 206 is equal to the number of the first plurality of convolutional filters in the first convolutional layer 218. As the number of convolutional filters in the first plurality of convolutional filters is greater the in the subsequent convolutional layers of reduced depth CNN 221, the number of feature maps in first plurality of feature maps is likewise greater than the number of feature maps in second plurality of feature maps 208 or third plurality of feature maps 210.

Second convolutional layer 220 receives as input the first plurality of feature maps 206, and identifies/extracts feature patterns therein using the second plurality of convolutional filters, to produce the second plurality of feature maps 208. Second convolutional layer 220 may comprise one or more convolutional filters, wherein receptive field size of the one or more convolutional filters of second convolutional layer 220 may be less than the threshold receptive field size.

Second plurality of feature maps 208 may comprise a plurality of output values produced by application of the one or more convolutional filters of the second convolutional layer 220 to the first plurality of feature maps 206.

Third convolutional layer 222 receives as input the second plurality of feature maps 208, and identifies/extracts feature patterns therein using the third plurality of convolutional filters, to produce the third plurality of feature maps 210. Third convolutional layer 222 may comprise one or more convolutional filters, wherein a receptive field size of the one or more convolutional filters of third convolutional layer 222 may be less than the threshold receptive field size.

Third plurality of feature maps 210 may comprise a plurality of output values produced by application of the one or more convolutional filters of the third convolutional layer 222 to the second plurality of feature maps 208. Third plurality of feature maps 210 are passed to classification layer 224.

Classification layer 224 receives as input third plurality of feature maps 210, and maps features represented therein to classification labels for each of the plurality of pixels of downsampled image 204. The classification labels may comprise labels indicating to which of a finite and pre-determined set of classes a given pixel most probably belongs, based on the learned parameters of reduced depth CNN 221. In one embodiment, classification layer 224 classifies each pixel of downsampled image 204 as either belonging to an ROI or as belonging to non-ROI. In some embodiments, reduced depth CNN 221 may produce segmentation maps comprising more than one type of ROI, and the classification labels output by classification layer 224 may comprise an indication of which type of ROI a pixel belongs, or if the pixel does not belong to an ROI. Classification layer 224 may comprise a softmax or other similar function known in the art of machine learning, which may receive as input one or more feature channels corresponding to a single location or sub-region of downsampled image 204, and which may output single most probable classification label for said location or sub-region.

The output of classification layer 224 comprises a matrix or array of pixel classifications, and is referred to as segmentation map 212. As can be seen in FIG. 2, segmentation map 212 visually indicates a region of downsampled image 204 corresponding to an ROI. The ROI indicated by segmentation map 212 is a kidney, however it will be appreciated that various other anatomical regions of interest or non-anatomical regions of interest may be segmented using a segmentation system such as segmentation system 200. The size/resolution of segmentation map 212 is substantially similar to the second size of downsampled image 204, and thus comprises a size/resolution substantially less than the first size/resolution of image 202.

Segmentation map 212 is upsampled, as indicated by upsampling 226, to produce upsampled segmentation map 214, wherein a size/resolution of upsampled segmentation map 214 is equal to the first size of image 202. Upsampling may comprise one or more known methods of image enlargement, such as max upsampling, minimum upsampling, average upsampling, bi-linear interpolation etc. In one embodiment, upsampling 226 may comprise applying one or more up-convolutional filters to segmentation map 212.

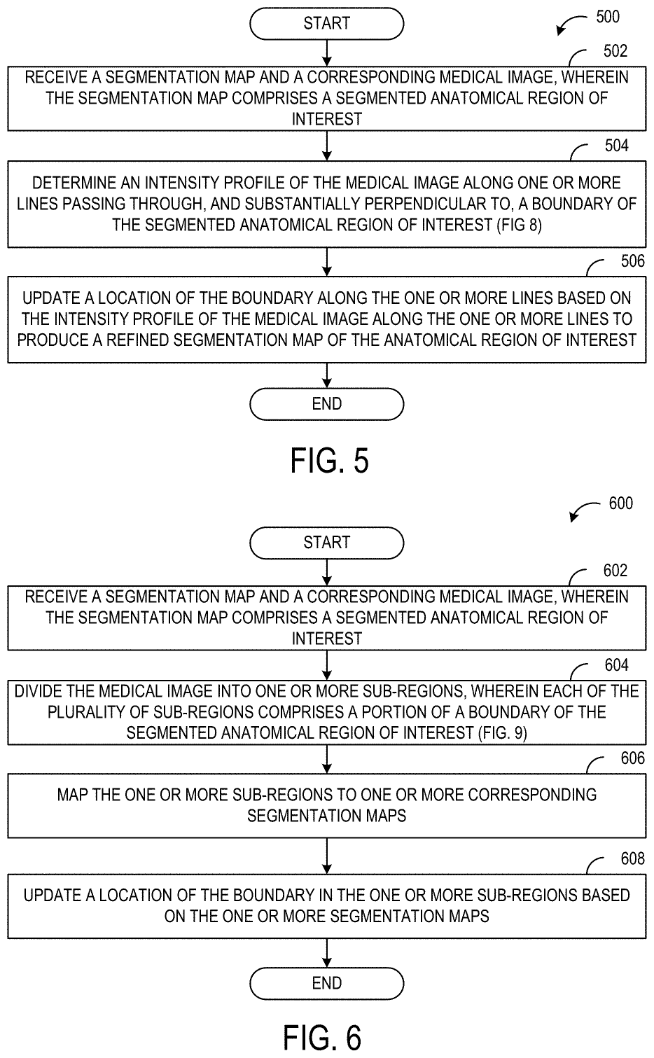

As segmentation map 212 was produced from downsampled data of downsampled image 204, upsampled segmentation map 214 may comprise pixelated/rough ROI boundaries, as can be seen in upsampled segmentation map 214. The inventors herein have identified systems and methods for refining rough/pixelated ROI boundary, which will be discussed in more detail with reference to FIGS. 5 and 6, below.

Thus, example segmentation system 200 illustrates one embodiment of a system which may receive an image and produce a segmentation map, using a reduced depth CNN 221 (with an associated reduction in training complexity/time and implementation complexity/time), wherein a depth of reduced depth CNN 221 may be substantially truncated compared to conventional CNNs (e.g., less than 6 convolutional layers), while preserving an accuracy of the segmentation map produced. As opposed to conventional approaches, image segmentation system 200 is able to directly learn (and thus subsequently identify) the structure of large features, comprising a majority of an extent of a region of interest in at least a first dimension (e.g., length, width, height, depth) in one convolution filter in the first convolutional layer, thus increasing the likelihood of identifying the ROI accurately and significantly reducing the dimensions of the network parameter optimization space, thus increasing the speed of training, inference, and reducing the chances of over fitting, and increasing the chances of converging to a parameter set which globally minimizes cost.

It should be understood that the architecture and configuration of reduced depth CNN 221 is for illustration, not for limitation. Other appropriate CNN architectures may be used herein for determining segmentation maps and/or image classifications without departing from the scope of the current disclosure. In particular, additional layers, including fully connected/dense layers, regularization layers, etc. may be used without departing from the scope of the current disclosure. Further, activation functions of various types known in the art of machine learning may be used following one or more convolutional layers and/or other layers. These described embodiments are only examples of systems and methods for determining segmentation maps using CNNs of reduced depth, the skilled artisan will understand that specific details described in the embodiments can be modified when being placed into practice without deviating from the spirit of the present disclosure.

Turning to FIG. 3, a flowchart of an example method 300 for producing segmentation maps of ROIs using a reduced depth CNN is shown. In one embodiment, image processing system 100, implementing segmentation system 200, may perform one or more operations of method 300, to produce a segmentation map of an ROI.

Method 300 may begin at operation 302, which includes the image processing system receiving an image having a first size. In some embodiments, the image may comprise a 2D or 3D image, or a time series of 2D or 3D images. In some embodiments the image comprises a medical image, acquired via a medical imaging device, and may include an anatomical region of interest to be segmented, such as an organ, tumor, implant, or other region of interest. The image may include metadata, indicating a type of anatomical region of interest captured by the medical image, what imaging modality was used to acquire the image, a size of the image, and one or more acquisition settings/parameters used during acquisition of the image.

At operation 304, the image processing system downsamples the image received at operation 302 to produce a downsampled image having a pre-determined, second size, wherein the second size is less than the first size. In some embodiments, the second size is less than 50% of the first size. The downsampling ratio may be determined dynamically based on a ratio between the first size and the pre-determined size. The downsampling may comprise pooling pixel intensity data, compressing pixel intensity data, and/or decimating pixel intensity data, of the image received at operation 302, to produce a downsampled image of the pre-determined size. In some embodiments, the pre-determined size may be dynamically selected from a list of pre-determined sizes, based on a ROI to be segmented and/or an indicated ROI included in the image. In some embodiments, the second size may be selected such that anatomical structures included therein retain sufficient resolution to be identified by a human observer.

At operation 306, the image processing system feeds the downsampled image produced at operation 304 to a trained reduced depth CNN, wherein a first convolutional layer of the trained reduced depth CNN comprises a first plurality of convolutional filters having receptive fields sizes larger than a threshold receptive field size. In embodiments where the input image received at operation 302 comprises a 2D image comprising 2D imaging data, the receptive field size threshold is a receptive field area threshold. In some embodiments, the receptive field area threshold comprises 5% to 100%, or any fractional amount therebetween, of the area of the pre-determined size of the downsampled image. In embodiments where the input image received at operation 302 comprises a 3D image comprising 3D imaging data, the receptive field size threshold is a receptive field volume threshold. In some embodiments, the receptive field volume threshold comprises 5% to 100%, or any fractional amount therebetween, of the volume of the pre-determined size of the downsampled image.

In some embodiments, the image processing system may select a trained reduced depth CNN from a plurality of trained reduced depth CNNs based on an ROI (or ROIs) for which the trained reduced depth CNN was trained to produce segmentation maps. The image processing system may determine which type(s) of ROI a trained reduced depth CNN is configured to segment based on metadata associated with the trained reduced depth CNN. In some embodiments, the threshold receptive field size of the trained reduced depth CNN is selected based on a desired ROI to be segmented, and further based on a shape/aspect ratio of the desired ROI. In some embodiments, the threshold receptive field size of the first plurality of convolutional filters in the first convolutional layer of the trained reduced depth CNN may be selected (prior to training) based on an expected/estimated fraction of coverage of the ROI in the downsampled image. In some embodiments, a first number of the first plurality of convolutional filters is greater than a number of convolutional filters in any one of the one or more subsequent layers of the trained reduced depth CNN. In some embodiments, the first number of the first plurality of convolutional filters is within the range of 100 to 3000, inclusive, or any integer therebetween. In some embodiments, none of the subsequent layers of the trained reduced depth CNN include a convolutional filter having a receptive field size greater than the threshold receptive field size. In some embodiments, the trained CNN comprises less than 6 convolutional layers. In some embodiments the receptive field size threshold may be 50% to 100% of the size of the downsampled image. In some embodiments, at least one dimension of a receptive field size threshold may be selected based on an anatomical region of interest to be segmented. In one embodiment, the threshold size comprises a threshold length, and wherein the threshold length is greater than 50% of a length of the anatomical region of interest in at least a first dimension/direction.

At operation 308, the image processing system identifies one or more features in the downsampled image using the first plurality of convolutional filters of the trained reduced depth CNN. As the receptive field sizes of each of the first plurality of convolutional filters in the first convolutional layer occupy a substantial portion of an expected ROI, the features extracted/identified at operation 308 may comprise substantial portions of ROIs present in the downsampled image. In some embodiments, the entirety of an ROI may be identified by a filter in the first convolutional layer of the trained reduced depth CNN. Briefly, filters identify/extract patterns by computing a dot product between the filter weights of a convolutional filter and the pixel intensity values of the downsampled image over a receptive field of the filter, the greater the magnitude of the dot product, the greater the degree of match between the filter and the pixel intensity pattern in the downsampled image. The dot product may be fed to an activation function, and then output to a feature map, which records serves to record the degree of match and spatial information of the region of the downsampled image where the match was found.

At operation 310, the image processing system maps the one or more identified features, identified at operation 308, to a segmentation map of one or more regions of interest using one or more subsequent layers of the trained CNN. In some embodiments, the segmentation map is of the second, predetermined size, equal to the size of the downsampled image produced at operation 304. The segmentation map may comprise a plurality of pixel classifications, corresponding to a number of pixels in the downsampled image, wherein each pixel classification provides a designation as to which of a finite and pre-determined set of classes a pixel of the downsampled image most probably belongs. In some embodiments, the one or more subsequent layers of the trained reduced depth CNN may comprise one or more additional convolutional layers, configured to receive the feature maps produced by the first convolutional neural network. In some embodiments, the receptive field sizes of convolutional filters in each of the subsequent convolutional layers is less than the threshold receptive field size, and a number of convolutional filters in each one of the subsequent layers is less than the number convolutional filters in the first convolutional layer.

At operation 312, the image processing system upsamples/enlarges the segmentation map, to produce an upsampled segmentation map having a size equal to the first size of the image received at operation 302. Upsampling may comprise one or more known methods of image enlargement such as adaptive or non-adaptive interpolation, including nearest neighbor approximation, bilinear interpolation, bicubic smoothing, bicubic sharpening, etc.

At operation 314, the image processing system refines ROI boundaries of the upsampled segmentation map based on the intensity values from the image received at operation 302, to produce a refined segmentation map. FIGS. 5 and 6 provide two distinct embodiments of methods for refining ROI boundaries, and are discussed in more detail below. Briefly, an ROI boundary is a location or region in a segmentation map where a pixel or cluster of pixels classified as belonging to an ROI touches one or more pixels classified as non-ROI. As can be seen by upsampled segmentation map 214 in FIG. 2, an ROI boundary (where the region of bright pixels contacts the region of black pixels) may be pixelated or rough, and may therefore provide an unclear designation of where an ROI ends. By upsampling the segmentation map produced at operation 310 to produce the upsampled segmentation matching the first size of the image received at operation 302, the original pixel intensity data of the image may be more efficiently leveraged to provide refined boundary locations for each ROI identified in the upsampled segmentation map, as there is a 1-to-1 correspondence between pixel labels in the upsampled segmentation map and pixels in the image. In particular, in methods 500 and 600, intensity values of the image are obtained from regions within a threshold distance of ROI boundaries identified in the upsampled segmentation map, thus intensity values within a threshold distance of ROI boundaries (which include higher resolution information than the upsampled segmentation map, which was produced from compressed/downsampled intensity data) may be used to more accurately locate a boundary between ROI and non-ROI regions.

At operation 316, method 300 optionally includes the image processing system displaying the refined segmentation map produced at operation 314 to a user via a display device.

At operation 318, method 300 optionally includes the image processing system determining one or more of a length, a width, a depth, a volume, a shape, and an orientation of the ROI based on the refined segmentation map. In some embodiments, the image processing system may employ principle component analysis of the refined segmentation map to determine one or more spatial parameters of the one or more segmented ROIs of the refined segmentation map. Following operation 318, method 300 may end.

In this way, method 300 may enable generation of segmentation maps using a reduced depth CNN, wherein the segmentation maps comprise ROI boundaries of substantially similar accuracy as those produced by computationally expensive conventional CNNs. As the reduced depth CNN comprises a smaller number of convolutional layers than in a conventional CNN, and thus a greatly reduced number of total network parameters, a segmentation map produced via implementation of method 300 may consume a fraction of the computational resources of a conventional CNN, and may be produced in a fraction of the time of a conventional CNN. A technical effect of setting a threshold receptive field size of convolutional filters in a first convolutional layer, based on an expected area/volume occupied by a desired ROI in a downsampled image, such that the convolutional filters in the first convolutional layer cover a majority of the ROI to be identified and segmented, is that the desired ROI may be identified using a substantially reduced number of convolutional filters. Further, a technical effect of downsampling an image prior to segmentation of one or more ROIs therein, upsampling a segmentation map produced from the downsampled image, and refining ROI boundaries in the upsampled segmentation map based on pixel intensity data from the original full sized image, is that a segmentation map of substantially similar accuracy as produced in conventional approaches, may be produced in a shorter duration of time employing reduced computational resources.

Turning now to FIG. 4, a flowchart of an example method 400 for classifying images using reduced depth CNNs is shown. Method 400 may be implemented by an image processing system, such as image processing system 100, to determine a standard view of an image. In some embodiments, method 400 may comprise determining to which of a finite number of standard views a medical image belongs. Briefly, in medical imaging, images of anatomical regions of interest are acquired in one of a finite number of orientations and/or with a pre-determined set of acquisition parameters, each distinct orientation/set of acquisition parameters is referred to as standard view, and in medical imaging workflows, identifying to which standard view a medical image belongs may inform downstream analysis and processing. Method 400 enables reduced computational complexity and increased classification speed, similar to the advantages obtained by method 300 in the case of image segmentation, but applied to image classification.

At operation 402, the image processing system receives an image comprising a standard view of an anatomical ROI. In some embodiments, the image processing system receives via wired or wireless communication with a medical imaging device a medical image comprising a standard view of an anatomical region of interest of an imaging subject. In some embodiments, the image may comprise a 2D or 3D image, or a time series of 2D or 3D images. The image may include metadata, indicating a type of anatomical region of interest captured by the image, what imaging modality was used to acquire the image, a size of the image, and one or more acquisition settings/parameters used during acquisition of the image.

At operation 404, the image processing system downsamples the image received at operation 402 to produce a downsampled image having a pre-determined size, wherein the pre-determined size is less than the first size. The downsampling ratio may be determined dynamically based on a ratio between the first size and the pre-determined size. The downsampling may comprise pooling pixel intensity data, compressing pixel intensity data, and/or decimating pixel intensity data, of the image received at operation 402, to produce a downsampled image of the pre-determined size. In some embodiments, the pre-determined size may be dynamically selected from a list of pre-determined sizes, based on one or more pieces of metadata associated with the image received at operation 402.

At operation 406, the image processing system feeds the downsampled image produced at operation 404 to a trained reduced depth CNN, wherein a first convolutional layer of the trained reduced depth CNN comprises a first plurality of convolutional filters having receptive fields sizes larger than 50% of the size (area or volume) of the downsampled image produced at operation 404. In other words, the receptive field size of each of the plurality of convolutional filters in the first convolutional layer of the trained reduced depth CNN are larger than a receptive field size threshold, wherein the receptive field size threshold is at least 50% of the area of the downsampled image produced at operation 404. In some embodiments, the image processing system may select a trained reduced depth CNN from a plurality of trained reduced depth CNNs based on one or more pieces of metadata associated with the image received at operation 402. In some embodiments, a first number of the first plurality of convolutional filters of the first convolutional layer is greater than a number of convolutional filters in any one of the one or more subsequent layers of the trained reduced depth CNN. In some embodiments, the first number of the first plurality of convolutional filters is within the range of 100 to 800, inclusive, or any integer therebetween. In some embodiments, none of the subsequent layers of the trained reduced depth CNN include a convolutional filter having a receptive field size greater than the threshold receptive field size. In some embodiments, the trained reduced depth CNN comprises less than 6 convolutional layers. In some embodiments, the trained reduced depth CNN comprises a single convolutional layer. In some embodiments the receptive field size threshold may be 50% to 100% of the size of the downsampled image. In some embodiments the receptive field size threshold may be at least 80% of the area/volume of a downsampled image.

At operation 408, the image processing system identifies one or more features in the downsampled image using the first plurality of convolutional filters of the trained reduced depth CNN. As the receptive field sizes of each of the first plurality of convolutional filters in the first convolutional layer occupy a substantial portion of the downsampled image (e.g., 50%-100%), the features extracted at operation 408 comprise more “holistic” features than are identified by a first convolutional layer of a conventional CNN. In particular, as opposed to conventional CNNs, where convolutional filters in a first convolutional layer may detect atomic features, such as lines, edges, corners, the features identified by the first layer of the trained reduced depth CNN may comprise “holistic” or “global” features, such as relative positioning of sub-regions within the downsampled image, orientations of anatomical features, overall image brightness, etc. Briefly, convolutional filters identify/extract patterns by computing a dot product between the filter weights of a convolutional filter and the pixel intensity values (or feature values if the input is a feature map) of the downsampled image over a receptive field of the filter, the greater the magnitude of the dot product, the greater the degree of match between the filter and the pixel intensity pattern in the downsampled image. The dot product may be fed to an activation function, and then output to a feature map, which serves to record the degree of match, and spatial information of the region of the downsampled image where the match was found.

At operation 410, the image processing system maps the one or more identified features, identified at operation 408, to an image classification of the downsampled image using one or more subsequent layers of the trained reduced depth CNN. In some embodiments, the one or more subsequent layers of the trained reduced depth CNN may comprise one or more additional convolutional layers, configured to receive the feature maps produced by the first convolutional layer. In some embodiments, the receptive field sizes of convolutional filters in each of the subsequent convolutional layers is less than the threshold receptive field size, and a number of convolutional filters in each one of the subsequent layers is less than the number convolutional filters in the first convolutional layer.

At operation 412, method 400 optionally includes the image processing device displaying a graphical user interface (GUI) via a display device, wherein the GUI is selected based on the image classification determined at operation 410. In some embodiments, image processing workflows may include displaying GUIs based on a standard view of an image, as an example, if an image classification indicates an image comprises a first anatomical region, imaged in a first orientation, a GUI comprising features/tools specific to analysis of the first anatomical region in the first orientation may be automatically displayed at operation 412, thus streamlining an image analysis and processing workflow. Following operation 412, method 400 may end.

A technical effect of using a reduced resolution/downsampled image, (in which the view type in the image is still recognizable), and then using a plurality of convolutional filters in the first layer of a reduced depth CNN having a receptive field size greater than 50% of the size of the input image, is that global orientational and positional features of sub-regions within the input image may be identified using a reduced number of convolutional layers, and thus the reduced depth CNN has higher accuracy and greater speed in classification of the standard view of the input image.

In this way, method 400 may enable fast and accurate detection of the standard view of acquired medical images. In some embodiments, method 400 may be employed in conjunction with real time image acquisition, to dynamically adjust a GUI displayed to a medical practitioner conducting a scan of anatomical regions of interest of a patient.

Turning to FIG. 5, a flowchart of a first embodiment of a method 500 for refining ROI boundaries of a segmentation map is shown. Method 500 may be implemented by image processing system 100 to increase accuracy of boundary locations of ROI boundaries of segmentation maps produced by reduced depth CNNs. As the reduced depth CNNs taught herein receive as input downsampled images, the corresponding resolution of output segmentation maps is also reduced, this increases the speed and computational efficiency of implementing such reduced depth CNNs, and by implementing one or more of the operations of methods 500 or 600, discussed below, an accuracy and smoothness of the ROI boundaries may be substantially equivalent to ROI boundaries produced using slower and less computationally efficient CNNs.

At operation 502, the image processing system receives a segmentation map (such as upsampled segmentation map 214) and a corresponding image (such as image 202), wherein the segmentation map comprises a segmented anatomical region of interest. In some embodiments, the segmentation map and image both are of a first size, such that for each pixel label of the segmentation map, there is a corresponding pixel at a corresponding location of the image. In some embodiments, the image processing system may receive the segmentation map and the image from a location of non-transitory memory, or from wired or wireless communication with a remotely located computing system.

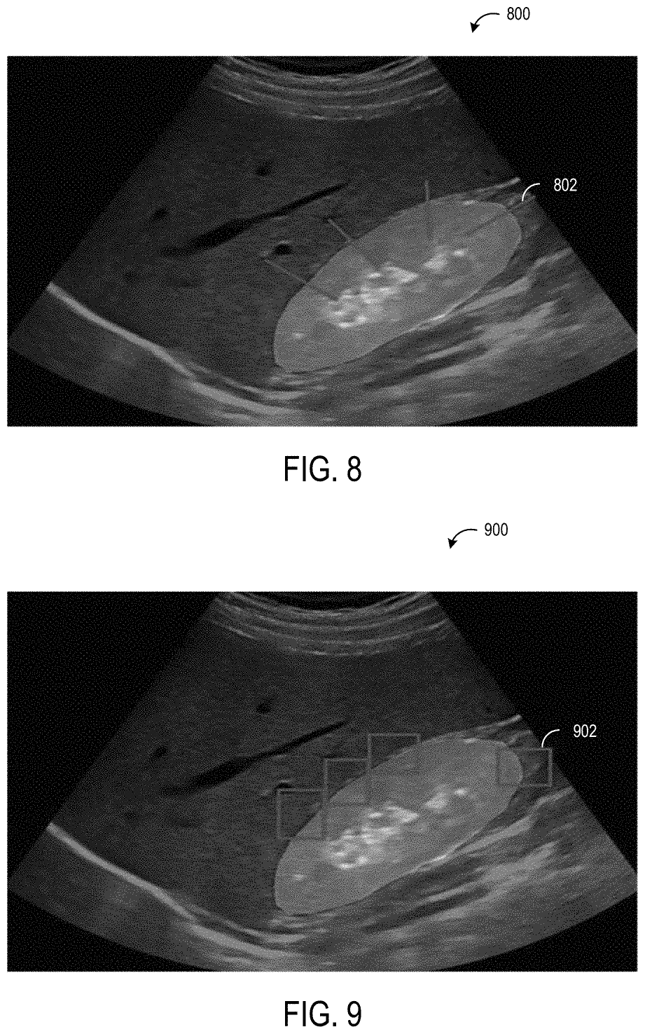

At operation 504, the image processing system determines an intensity profile of the medical image along one or more lines passing through, and substantially perpendicular to, a boundary of the segmented anatomical region of interest, wherein the one or more lines are each substantially bisected by the ROI boundary (that is, a center of the one or more lines each substantially coincides/intersects with a portion of an ROI boundary). Turning briefly to FIG. 8, an illustration of the concept of operation 504 is shown. As can be seen in FIG. 8, a plurality of intensity profiles, such as intensity profile 802, are sampled along lines of threshold length, passing substantially perpendicular to a boundary of a segmented ROI. The position of each of the one or more lines is determined using the segmentation map, and then pixel intensity values are sampled from the image at corresponding locations. The number of lines employed may be pre-selected based on an expected shape/geometry of the ROIs to be segmented. As an example, as the expected complexity of a boundary of a ROI increases, the number of lines employed at operation 504 may correspondingly increase. The threshold length of the lines may be selected based on a desired degree of computational complexity, with longer lines corresponding to increased computational complexity, and with shorter lines corresponding to reduced computational complexity. Further, the threshold length of the lines may be selected based on a size of downsampled image used to produce the segmentation map, wherein as the size of the downsampled image from which the segmentation map is produced decreases, a length of the lines of operation 504 may increase to compensate for the increased pixilation which may arise in upsampled segmentation maps produced thereby. In embodiments where the segmentation map comprises a 3D segmentation map, the lines of method 500 and FIG. 8 may be replaced with planes, wherein substantially half of the area of the planes are within the ROI and substantially half of the area of the planes are outside of the ROI.

At operation 506, the image processing system updates a location of the ROI boundary along each of the one or more lines based on the intensity profile of the image along the one or more lines, to produce a refined segmentation map of the anatomical region of interest. In some embodiments, the one or more lines extends from a threshold distance inside of the ROI boundary to a threshold distance outside of the ROI boundary. In some embodiments, one or more conventional edge detection algorithms may be employed to determine an updated ROI boundary location along each of the one or more lines, using the corresponding intensity profiles of the one or more lines obtained from the original full-sized image. Briefly, edge detection algorithms may evaluate changes or discontinuities in pixel intensity data along the one or more lines to determine an updated ROI boundary location. In some embodiments, each of the intensity profiles along each of the one or more lines may be fed to a trained neural network, trained to map one dimensional intensity vectors to edge locations.

In this way, method 500 may enable pixelated ROI boundaries in a segmentation map produced by a reduced depth CNN to be converted into smooth and accurate ROI boundaries, by leveraging pixel intensity data from an intelligently selected subset of regions from within an original full-sized image from which the segmentation map was produced.

Turning to FIG. 6, a flowchart of a second embodiment of a method 600 for refining boundaries of ROIs in segmentation maps produced by reduced depth CNNs is shown. Method 600 may be implemented by an image processing system, such as image processing system 100, to increase the smoothness and accuracy of ROI boundaries in segmentation maps produced by reduced depth CNNs.

At operation 602, the image processing system receives a segmentation map (such as upsampled segmentation map 214) and a corresponding image (such as image 202), wherein the segmentation map comprises a segmented anatomical region of interest. In some embodiments, the segmentation map and image both are of a first size, such that for each pixel label of the segmentation map, there is a corresponding pixel at a corresponding location of the image. In some embodiments, the image processing system may receive the segmentation map and the image from a location of non-transitory memory, or from wired or wireless communication with a remotely located computing system.

At operation 604, the image processing system divides the medical image into one or more sub-regions, wherein each of the plurality of sub-regions comprises a portion of a boundary of the segmented anatomical region of interest. Turning briefly to FIG. 9, an illustration of the concept of operation 604 is shown. As can be seen in FIG. 9, a plurality of sub-regions of pixel intensity values, such as sub-region 902, are sampled from the image received at operation 602. In some embodiments the sub-regions are square or rectangular, in other embodiments the sub regions may be circular or oblong. Each of the sub-regions may be of threshold area, wherein substantially half of the area of the sub-regions are within the ROI (inside of the initially estimated ROI boundary of the upsampled segmentation map) and substantially half of the area of the planes are outside of the ROI (outside of the initially estimated ROI boundary of the upsampled segmentation map received at operation 602). The position of each of the sub-regions is determined using the segmentation map received at operation 602, and then pixel intensity values are sampled from the image received at operation 602 at locations corresponding to the sub-regions. The number of sub-regions employed may be pre-selected based on an expected shape/geometry of the ROIs to be segmented. As an example, as the expected complexity of a boundary of a ROI increases, the number of sub-regions employed at operation 604 may correspondingly increase, and the size/area of coverage of the sub-regions may correspondingly decrease. In embodiments where the segmentation map comprises a 3D segmentation map, the sub-region of method 600 and FIG. 9 may be replaced with volumic sub-regions, such as cubes, rectangular solids, spheres, etc. planes, wherein substantially half of the volume 3D sub-regions are within the ROI and substantially half of the volume of the 3D sub-regions are outside of the ROI.

At operation 606, the image processing system feeds the one or more sub-regions to a trained CNN, wherein the trained CNN is configured to map matrices of pixel intensity values corresponding, to each of the sub-regions, to a corresponding edge segmentation map, indicating an updated position of an ROI boundary along a line (or plane in the case of 3D images and 3D segmentation maps) for each of the sub-regions. In some embodiments, identification of ROI boundaries within the one or more sub-regions may comprise one or more conventional edge detection algorithms.

At operation 608, the image processing system updates a location of the ROI boundary in the one or more sub-regions based on the one or more segmentation maps produced at operation 606. Following operation 608, method 600 may end. In this way, method 600 enables pixelated ROI boundaries in a segmentation map produced by a reduced depth CNN to be converted into smooth and accurate ROI boundaries, by leveraging pixel intensity data from an intelligently selected subset of regions from within an original full-sized image from which the segmentation map was produced.

Turning to FIG. 7, a flowchart of an example method 700 for training a reduced depth CNN (such as reduced depth CNN 221, shown in FIG. 2) to infer a segmentation map of one or more ROIs from an input downsampled image, is shown. Method 700 may be executed by one or more of the systems discussed above. In some embodiments, method 700 may be implemented by image processing system 100 shown in FIG. 1. In some embodiments, method 700 may be implemented by training module 112, stored in non-transitory memory 106 of image processing device 102.

At operation 702, a training data pair, from a plurality of training data pairs, is fed to an input layer of a reduced depth CNN, wherein the training data pair comprises an image and a corresponding ground truth segmentation map. The training data pair may be intelligently selected by the image processing system based on one or more pieces of metadata associated with the training data pair. In one embodiment, method 700 may be employed to train a reduced depth CNN to identify one or more pre-determined types of ROIs, and operation 702 may include the image processing system selecting a training data pair comprising an image, wherein the image includes one or more of the pre-determined types of ROIs, and wherein the training data pair further comprising a ground truth segmentation map of the one or more ROIs in the image. In some embodiments, the ground truth segmentation maps may be produced by an expert, such as by a radiologist.

In some embodiments, the training data pair, and the plurality of training data pairs, may be stored in an image processing device, such as in image data module 114 of image processing device 102. In other embodiments, the training data pair may be acquired via communicative coupling between the image processing system and an external storage device, such as via Internet connection to a remote server.

At operation 704, the image of the training data pair is mapped to a predicted segmentation map using the reduced depth CNN. In some embodiments, operation 704 may comprise inputting pixel/voxel intensity data of the image into an input layer of the reduced depth CNN, identifying features present in the image using at least a first convolutional layer comprising a first plurality of convolutional filters, wherein each of the plurality of convolutional filters comprises a receptive field size greater than a threshold receptive field size, and mapping the features extracted by the first convolutional layer to the predicted segmentation map using one or more subsequent layers. In some embodiments, the one or more subsequent layers comprise at least a classification layer.

At operation 706, the image processing system calculates a loss for the reduced depth CNN based on a difference between the predicted segmentation map and the ground truth segmentation map. Said another way, operation 706 comprises the image processing system determining an error of the predicted segmentation map using the ground-truth segmentation map, and a loss/cost function. In some embodiments, operation 706 includes the image processing system determining a plurality of pixel classification label differences between a plurality of pixels/voxels of the predicted segmentation map and a plurality of pixels/voxels of the ground-truth segmentation map, and inputting the plurality of pixel classification label differences into a pre-determined loss/cost function (e.g., an MSE function, or other loss function known in the art of machine learning). In some embodiments, the loss function may comprise a DICE score, a mean square error, an absolute distance error, or a weighted combination of one or more of the preceding. In some embodiments, operation 706 may comprise determining a DICE score for the predicted segmentation map using the ground-truth segmentation map according to the following equation:

DICE=(S∩T)/(S∪T),

wherein S is the ground-truth segmentation map, and wherein T is the predicted segmentation map.

At operation 708, the weights and biases of the reduced depth CNN are updated based on the loss determined at operation 706. In some embodiments, the loss back propagated through the layers of the reduced depth CNN, and the parameters of the reduced depth CNN may be updated according to a gradient descent algorithm based on the back propagated loss. The loss, may be back propagated through the layers of the reduced depth CNN to update the weights (and biases) of each of the layers. In some embodiments, back propagation of the loss may occur according to a gradient descent algorithm, wherein a gradient of the loss function (a first derivative, or approximation of the first derivative) is determined for each weight and bias of the reduced depth CNN. Each weight (and bias) of the reduced depth CNN is then updated by adding the negative of the product of the gradient determined (or approximated) for the weight (or bias) and a predetermined step size, according to the below equation:

$${P}_{i + 1} = {P}_{i} - Step \frac{\partial ( loss )}{\partial {P}_{i}}$$

Where Pᵢ₊₁ is the updated parameter value, Pᵢ is the previous parameter value, Step is the step size, and

$$\frac{\partial ( loss )}{\partial {P}_{i}}$$

is the partial derivative of the loss with respect to the previous parameter.

Following operation 708, method 600 may end. It will be noted that method 700 may be repeated until the weights and biases of the reduced depth CNN converge, a threshold loss is obtained (for the training data or on a separate validation dataset), or the rate of change of the weights and/or biases of the reduced depth CNN for each iteration of method 700 are under a threshold rate of change. In this way, method 700 enables a reduced depth CNN to be trained to infer segmentation maps for one or more ROIs from downsampled images.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method comprising:
receiving an image having a first size;
downsampling the image to produce a downsampled image of a pre-determined size, wherein the pre-determined size is less than the first size;
feeding the downsampled image to a convolutional neural network (CNN), wherein a first convolutional layer of the CNN comprises a first plurality of convolutional filters, each of the first plurality of convolutional filters having a receptive field size larger than a threshold receptive field size;
identifying one or more anatomical structures of the downsampled image using the first plurality of convolutional filters; and
mapping the one or more anatomical structures to a segmentation map or image classification using one or more subsequent layers of the CNN.

2. The method of claim 1, wherein the receptive field size threshold is from 5% to 100% of the predetermined size.

3. The method of claim 1, wherein the pre-determined size is less than 50% of the first size.

4. The method of claim 1, wherein a first number of the first plurality of convolutional filters is within a range of 100 to 3000, inclusive, or any integer therebetween.

5. The method of claim 1, wherein none of the one or more subsequent layers have input size smaller than the pre-determined size.

6. The method of claim 1, wherein the image comprises two-dimensional imaging data of an anatomical region of an imaging subject, and the threshold receptive field size is a receptive field area threshold.

7. The method of claim 1, wherein the image comprises three-dimensional imaging data of an anatomical region of an imaging subject, and the threshold receptive field size is a receptive field volume threshold.

8. The method of claim 1, wherein downsampling the image comprises determining a ratio of the first size to the pre-determined size, and dynamically determining a downsampling ratio based on the ratio of the first size to the pre-determined size.

9. The method of claim 1, wherein the image classification comprises an indication of a standard view of the image, the method further comprising:
selecting a graphical user interface (GUI) based on the standard view; and
displaying the GUI via a display device.

10. The method of claim 1, wherein the image comprises a medical image including an anatomical region of interest, and wherein the segmentation map comprises a segmentation map of the anatomical region of interest.

11. The method of claim 10, wherein the threshold receptive field size comprises a threshold area or volume, and wherein the threshold area or volume is greater than 20% of an area or volume occupied by the anatomical region of interest in the downsampled image.

12. An image processing system, comprising:
a memory storing a convolutional neural network (CNN), and instructions; and
a processor, wherein the processor is communicably coupled to the memory, and when executing the instructions, configured to:
receive a two-dimensional image or three-dimensional image, of a first size;
determine a downsampling ratio based on the first size and a pre-determined size;
downsample the image using the downsampling ratio to produce a downsampled image of the pre-determined size, wherein the pre-determined size is less than the first size;
feed the downsampled image to the CNN, wherein a first convolutional layer of the CNN comprises a first plurality of convolutional filters, each of the first plurality of convolutional filters having a receptive field size larger than a threshold size;
identify one or more anatomical structures of the downsampled image using the first plurality of convolutional filters; and
map the one or more anatomical structures to an output using one or more subsequent layers of the CNN, wherein none of the one or more subsequent layers include a pooling operation.

13. The image processing system of claim 12, wherein the output comprises a segmentation map of an anatomical region of interest, and wherein, when executing the instructions, the processor is further configured to:
upsample the segmentation map to produce an upsampled segmentation map of the first size; and
refine a boundary of the upsampled segmentation map based on intensity values of the image within a threshold distance of a boundary of the anatomical region of interest to produce a refined segmentation map.

14. The image processing system of claim 13, further comprising a display device, and wherein, when executing the instructions, the processor is further configured to:
display the refined segmentation map via the display device.

15. The image processing system of claim 12, wherein the output comprises an image classification, indicating to which standard view of a finite list of standard views the image belongs.

16. A method comprising:
receiving a medical image comprising an anatomical region of interest, wherein the medical image is of a first size;
determining a downsampling ratio based on the first size and a pre-determined size;
downsampling the medical image using the downsampling ratio to produce a downsampled image of the pre-determined size, wherein the pre-determined size is less than 50% of the first size;
feeding the downsampled image to a convolutional neural network (CNN), wherein a first convolutional layer of the CNN comprises a first plurality of convolutional filters, each of the first plurality of convolutional filters having a receptive field configured to receive data from a pre-determined fraction of the downsampled image, wherein the pre-determined fraction is from 5% to 100% of the area or volume of the downsampled image;
identifying one or more features of the downsampled image using the first plurality of convolutional filters; and
mapping the one or more features to an output using one or more subsequent layers of the CNN.

17. The method of claim 16, wherein the output comprises a two-dimensional or three-dimensional segmentation map of the anatomical region of interest, the method further comprising:
upsampling the segmentation map to produce an upsampled segmentation map;
refining a boundary of the anatomical region of interest in the upsampled segmentation map to produce a refined segmentation map; and
determining one or more of a length, a width, a shape, and an orientation of the anatomical region of interest based on the refined segmentation map.

18. The method of claim 17, wherein refining the boundary of the anatomical region of interest in the upsampled segmentation map comprises:
determining a plurality of intensity profiles of the medical image along a plurality of lines passing through, and substantially perpendicular to, the boundary of the anatomical region of interest; and
updating a location of the boundary of the anatomical region of interest in the upsampled segmentation map based on the plurality of intensity profiles.

19. The method of claim 18, wherein updating the location of the boundary of the anatomical region of interest in the upsampled segmentation map based on the plurality of intensity profiles comprises:
mapping each of the plurality of intensity profiles to a corresponding boundary location using a trained neural network; and
updating the location of the boundary along each of the plurality of lines to the corresponding boundary location.

20. The method of claim 17, wherein refining the boundary of the anatomical region of interest in the upsampled segmentation map comprises:
dividing the medical image into a plurality of sub-regions, wherein each of the plurality of sub-regions comprises a portion of the boundary of the anatomical region of interest;
mapping each of the plurality of sub-regions to a corresponding segmentation map using a second trained convolutional neural network; and
updating the location of the boundary within each of the plurality of sub-regions based on the corresponding segmentation map.
